(19) United States
(12) Patent Application Publication
(10) Pub. No.: US 2022/0361787 A1
(43) Pub. Date: Nov. 17, 2022

(54) EAR-WORN DEVICE BASED MEASUREMENT OF REACTION OR REFLEX SPEED

(71) Applicant: Starkey Laboratories, Inc., Eden Prairie, MN (US)

(72) Inventors: Justin R. Burwinkel, Eden Prairie, MN (US); Buye Xu, Sammamish, WA (US); Sourav K. Bhunia, Shoreview, MN (US); Jason A. Galster, Studio City, CA (US); Jing Xia, San Jose, CA (US); Lauren Petley, Cincinnati, OH (US)

(21) Appl. No.: 17/628,436

(22) PCT Filed: Jul. 17, 2020
(86) PCT No.: PCT/US2020/042571

§ 371 Date: Jan. 19, 2022

Related U.S. Application Data
(60) Provisional application No. 62/876,458, filed on Jul. 19, 2019.

(51) Int. Cl. A61B 5/16; A61B 5/00; A61B 5/11

(52) U.S. Cl. CPC A61B 5/162; A61B 5/7275; A61B 5/0051; A61B 5/6817; A61B 5/1114; A61B 5/6898; A61B 5/1104; A61B 5/746; A61B 5/4023; A61B 2562/0204; A61B 2562/0219; A61B 5/398

(57) ABSTRACT

Embodiments herein relate to ear-worn devices and, more specifically, ear-worn devices that can measure reaction and/or reflex speeds. An ear-worn device herein can include a control circuit, a clock circuit in electrical communication with the control circuit, a motion sensor in electrical communication with the control circuit, an electroacoustic transducer for generating sound in electrical communication with the control circuit, and a power supply circuit in electrical communication with the control circuit. The ear-worn device can be configured to initiate generation of a stimulus sufficient to generate a response from the ear-worn device wearer. The ear-worn device can be configured to monitor for a qualified response to the stimulus and measure an amount of time between the stimulus and the qualified response. Other embodiments are also included herein.

This application is being filed as a PCT International Patent application on Jul. 17, 2020 in the name of Starkey Laboratories, Inc., a U.S. national corporation, applicant for the designation of all countries, and Justin R. Burwinkel, a U.S. Citizen, and Buye Xu, a Citizen of China, and Sourav K. Bhunia, a U.S. Citizen, and Jason A. Galster, a U.S. Citizen, and Jing Xia, a U.S. Citizen, and Lauren Petley, a Citizen of Canada, inventors for the designation of all countries, and claims priority to U.S. Provisional Patent Application No. 62/876,458 filed Jul. 19, 2019, the contents of which are herein incorporated by reference in its entirety.

### FIELD

Embodiments herein relate to ear-worn devices and, more specifically, ear-worn devices that can measure reaction and/or reflex speeds.

### BACKGROUND

Reaction time is a measure of the speed of response to a stimulus. Reaction time has many practical implications. For example, a slow reaction time may make a subject more prone to falls. A slow reaction time may also make for a less safe driver. Conversely, fast reaction times be a benefit in sports.

Factors that can affect human reaction time include various factors including age, sex, left or right handedness, practice, fatigue, fasting, breathing cycle, and exercise. Generally, however, cognitive function/status of the subject greatly impacts reaction time. Therefore, reaction time is an important metric for gauging a subject's cognitive function/status.

### SUMMARY

Embodiments herein relate to ear-worn devices and, more specifically, ear-worn devices that can measure reaction and/or reflex speeds. In a first aspect, an ear-worn device can include a control circuit, a clock circuit in electrical communication with the control circuit, a motion sensor in electrical communication with the control circuit, an electroacoustic transducer for generating sound in electrical communication with the control circuit, and a power supply circuit in electrical communication with the control circuit. The ear-worn device can be configured to initiate generation of a stimulus sufficient to generate a response from the ear-worn device wearer. The ear-worn device can be configured to monitor for a qualified response to the stimulus and measure an amount of time between the stimulus and the qualified response.

In a second aspect, in addition to one or more of the preceding or following aspects, or in the alternative to some aspects, a motion sensor can be included and can be in electrical communication with the control circuit.

In a third aspect, in addition to one or more of the preceding or following aspects, or in the alternative to some aspects, a microphone can be included and can be in electrical communication with the control circuit.

In a fourth aspect, in addition to one or more of the preceding or following aspects, or in the alternative to some aspects, the stimulus can include an auditory stimulus.

In a fifth aspect, in addition to one or more of the preceding or following aspects, or in the alternative to some aspects, the stimulus can include an auditory stimulus generated by the electroacoustic transducer.

In a sixth aspect, in addition to one or more of the preceding or following aspects, or in the alternative to some aspects, the stimulus can include audible words.

In a seventh aspect, in addition to one or more of the preceding or following aspects, or in the alternative to some aspects, the words can be degraded.

In an eighth aspect, in addition to one or more of the preceding or following aspects, or in the alternative to some aspects, the words can be time-compressed.

In a ninth aspect, in addition to one or more of the preceding or following aspects, or in the alternative to some aspects, the stimulus further can include competing noise, vocoded speech, and frequency attenuation.

In a tenth aspect, in addition to one or more of the preceding or following aspects, or in the alternative to some aspects, the stimulus can include a tactile stimulus.

In an eleventh aspect, in addition to one or more of the preceding or following aspects, or in the alternative to some aspects, the stimulus can include a game event.

In a twelfth aspect, in addition to one or more of the preceding or following aspects, or in the alternative to some aspects, a game event can be generated by a device in electronic communication with the ear-worn device.

In a thirteenth aspect, in addition to one or more of the preceding or following aspects, or in the alternative to some aspects, the stimulus can include an action from an external device requesting a response from the ear-worn device wearer.

In a fourteenth aspect, in addition to one or more of the preceding or following aspects, or in the alternative to some aspects, an action can include a ringer sound, a message notification, or a query.

In a fifteenth aspect, in addition to one or more of the preceding or following aspects, or in the alternative to some aspects, the stimulus can include a dual-task paradigm stimulus.

In a sixteenth aspect, in addition to one or more of the preceding or following aspects, or in the alternative to some aspects, the ear-worn device can be configured to monitor for a qualified response using the motion sensor.

In a seventeenth aspect, in addition to one or more of the preceding or following aspects, or in the alternative to some aspects, the qualified response can include a reaction motion.

In an eighteenth aspect, in addition to one or more of the preceding or following aspects, or in the alternative to some aspects, the qualified response can include a post-auricular reflex or activation of periauricular muscles.

In a nineteenth aspect, in addition to one or more of the preceding or following aspects, or in the alternative to some aspects, the qualified response can include a balance recovery event.

In a twentieth aspect, in addition to one or more of the preceding or following aspects, or in the alternative to some aspects, the ear-worn device can be configured to monitor for the qualified response using a microphone, an EOG sensor, or an EEG sensor.

In a twenty-first aspect, in addition to one or more of the preceding or following aspects, or in the alternative to some aspects, the ear-worn device can be configured to monitor for the qualified response using a sensor attached to a separate device.

In a twenty-second aspect, in addition to one or more of the preceding or following aspects, or in the alternative to some aspects, the ear-worn device can be configured to evaluate the measured amount of time between the stimulus and the qualified response longitudinally and determine longitudinal trends.

In a twenty-third aspect, in addition to one or more of the preceding or following aspects, or in the alternative to some aspects, the ear-worn device can issue an alert in response to a determined longitudinal trend crossing a threshold value.

In a twenty-fourth aspect, in addition to one or more of the preceding or following aspects, or in the alternative to some aspects, the ear-worn device can be configured to determine typical changes in the amount of time between the stimulus and the qualified response for the ear-worn device wearer.

In a twenty-fifth aspect, in addition to one or more of the preceding or following aspects, or in the alternative to some aspects, the ear-worn device can be configured to compare the amount of time between the stimulus and the qualified response for the ear-worn device wearer to an average amount of time between stimuli and qualified responses for a population ear-worn device wearers.

In a twenty-sixth aspect, in addition to one or more of the preceding or following aspects, or in the alternative to some aspects, the ear-worn device can be configured to determine typical amounts of time between the stimulus and the qualified response for a population of ear-worn device wearers based on a type of stimulus.

In a twenty-seventh aspect, a method of measuring a response time of a hearing device wearer is included, the method can include initiating the provision of a stimulus to the hearing device wearer with an ear-worn device. The ear-worn device can include a control circuit, a clock circuit in electrical communication with the control circuit, an electroacoustic transducer for generating sound in electrical communication with the control circuit, and a power supply circuit in electrical communication with the control circuit. The method can further include monitoring for a qualified response to the stimulus using at least one of a motion sensor and a microphone.

In a twenty-eighth aspect, in addition to one or more of the preceding or following aspects, or in the alternative to some aspects, initiating the provision of a stimulus to the hearing device wearer can include delivering an auditory stimulus to the hearing device wearer.

In a twenty-ninth aspect, in addition to one or more of the preceding or following aspects, or in the alternative to some aspects, initiating the provision of a stimulus to the hearing device wearer can include delivering a tactile stimulus to the hearing device wearer.

In a thirtieth aspect, in addition to one or more of the preceding or following aspects, or in the alternative to some aspects, initiating the provision of a stimulus to the hearing device wearer can include delivering a visual stimulus to the hearing device wearer.

In a thirty-first aspect, in addition to one or more of the preceding or following aspects, or in the alternative to some aspects, initiating the provision of a stimulus to the hearing device wearer can include delivering an electrical stimulus to the hearing device wearer.

In a thirty-second aspect, in addition to one or more of the preceding or following aspects, or in the alternative to some aspects, the qualified response can include motion detected with the motion sensor.

In a thirty-third aspect, in addition to one or more of the preceding or following aspects, or in the alternative to some aspects, the motion sensor can be disposed within an ear worn device.

In a thirty-fourth aspect, in addition to one or more of the preceding or following aspects, or in the alternative to some aspects, the qualified response can include a reaction motion.

In a thirty-fifth aspect, in addition to one or more of the preceding or following aspects, or in the alternative to some aspects, the qualified response can include a post-auricular reflex or activation of periauricular muscles.

In a thirty-sixth aspect, in addition to one or more of the preceding or following aspects, or in the alternative to some aspects, the qualified response can include a balance recovery event.

In a thirty-seventh aspect, in addition to one or more of the preceding or following aspects, or in the alternative to some aspects, the qualified response can include sound detected with the microphone exceeding a threshold value.

In a thirty-eighth aspect, in addition to one or more of the preceding or following aspects, or in the alternative to some aspects, the method can further include using the measured response time to calculate a fall risk value or a fall risk threshold.

In a thirty-ninth aspect, in addition to one or more of the preceding or following aspects, or in the alternative to some aspects, a method can include changing at least one of a hearing device configuration or a signal processing setting and using the measured response time to determine if the change benefits the device wearer, wherein a decrease in the measured response time over a previously measured response time is indicative of a benefit to the device wearer.

In a fortieth aspect, in addition to one or more of the preceding or following aspects, or in the alternative to some aspects, a method can further include measuring the response time at a plurality of time points following at least one event; determining whether the event has caused an improvement, a decline, or no change to the device wearer, wherein a longitudinal decrease in the measured response time is indicative of an improvement.

In a forty-first aspect, an ear-worn device can be included herein and can include a control circuit; a clock circuit in electrical communication with the control circuit; an electroacoustic transducer for generating sound in electrical communication with the control circuit; a power supply circuit in electrical communication with the control circuit. The ear-worn device can be configured to detect a stimulus sufficient to generate a response from the ear-worn device wearer, monitor for a qualified response to the stimulus, and measure an amount of time between the stimulus and the qualified response.

In a forty-second aspect, in addition to one or more of the preceding or following aspects, or in the alternative to some aspects, the stimulus can include a detected auditory, tactile, or visual stimulus.

In a forty-third aspect, in addition to one or more of the preceding or following aspects, or in the alternative to some aspects, the qualified response can include a signal from a microphone.

In a forty-fourth aspect, in addition to one or more of the preceding or following aspects, or in the alternative to some aspects, the stimulus can include the ear-worn device wearer's name as detected with the microphone.

In a forty-fifth aspect, in addition to one or more of the preceding or following aspects, or in the alternative to some aspects, the stimulus can include an utterance matching an individual's voice selected from a group of predetermined individuals familiar to the device wearer.

In a forty-sixth aspect, in addition to one or more of the preceding or following aspects, or in the alternative to some aspects, the stimulus can include an action from an external device requesting a response from the ear-worn device wearer.

In a forty-seventh aspect, in addition to one or more of the preceding or following aspects, or in the alternative to some aspects, the action can include a ringer sound, a message notification, or a query.

In a forty-eighth aspect, in addition to one or more of the preceding or following aspects, or in the alternative to some aspects, the qualified response can include a signal from a motion sensor.

In a forty-ninth aspect, in addition to one or more of the preceding or following aspects, or in the alternative to some aspects, the qualified response can include a signal from the motion sensor indicative of at least one of eye movement, head movement, or a body movement.

In a fiftieth aspect, in addition to one or more of the preceding or following aspects, or in the alternative to some aspects, the qualified response can include a signal from the motion sensor indicative of the ear-worn device wearer turning their head toward the direction of the stimulus.

In a fifty-first aspect, in addition to one or more of the preceding or following aspects, or in the alternative to some aspects, the ear-worn device can be configured to monitor for the qualified response using a sensor attached to a separate device.

In a fifty-second aspect, in addition to one or more of the preceding or following aspects, or in the alternative to some aspects, the ear-worn device can be configured to evaluate the measured amount of time between the stimulus and the qualified response longitudinally and determine longitudinal trends.

In a fifty-third aspect, in addition to one or more of the preceding or following aspects, or in the alternative to some aspects, the ear-worn device can issue an alert in response to a determined longitudinal trend crossing a threshold value.

In a fifty-fourth aspect, in addition to one or more of the preceding or following aspects, or in the alternative to some aspects, the ear-worn device can be configured to determine typical changes in the amount of time between the stimulus and the qualified response for the ear-worn device wearer.

In a fifty-fifth aspect, in addition to one or more of the preceding or following aspects, or in the alternative to some aspects, the ear-worn device can be configured to compare the amount of time between the stimulus and the qualified response for the ear-worn device wearer to an average amount of time between stimuli and qualified responses for a population ear-worn device wearers.

In a fifty-sixth aspect, in addition to one or more of the preceding or following aspects, or in the alternative to some aspects, the ear-worn device can be configured to determine typical amounts of time between the stimulus and the qualified response for a population of ear-worn device wearers based on a type of stimulus.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a flowchart of operations of various devices herein is shown in accordance with various embodiments herein.

FIG. 2 is a diagram showing types of stimuli used in accordance with various embodiments herein.

FIG. 3 is a diagram showing some aspects that can be sensed in accordance with various embodiments herein.

FIG. 4 is a diagram showing aspects of response evaluation in accordance with various embodiments herein.

FIG. 5 is a schematic view of an ear-worn device in accordance with various embodiments herein.

FIG. 6 is a partial cross-sectional view of ear anatomy.

FIG. 7 is a schematic view of an ear-worn device disposed within the ear of a subject in accordance with various embodiments herein.

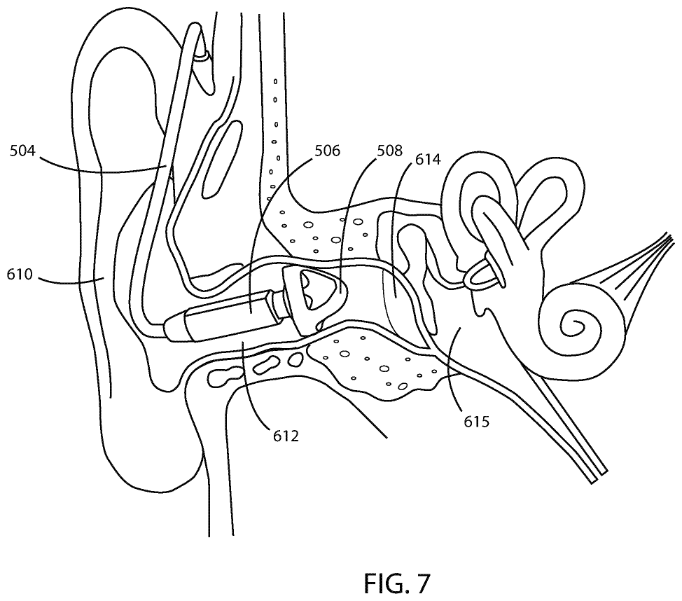

FIG. 8 is a schematic side view of a subject wearing an ear-worn device in accordance with various embodiments herein.

FIG. 9 is a schematic top view of a subject wearing ear-worn devices in accordance with various embodiments herein.

FIG. 10 is a schematic view of a device wearer interfacing with an external device in accordance with various embodiments herein.

FIG. 11 is a schematic frontal view of a subject wearing ear-worn devices in accordance with various embodiments herein.

FIG. 12 is a schematic side view of a subject wearing an ear-worn device in accordance with various embodiments herein.

FIG. 13 is a schematic view of data and/or signal flow as part of a system in accordance with various embodiments herein.

FIG. 14 is a schematic block diagram of various components of an ear-worn device in accordance with various embodiments.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, reaction time is an important metric for gauging an individual's cognitive status. However, it is not widely measured outside of research settings. This is because of practical challenges associated with performing such measurements.

However, in accordance with embodiments herein, ear-worn devices can be used to measure reaction and/or reflex speeds. By way of example, in some embodiments an ear-worn device can be configured to monitor for a qualified response to a stimulus and measure the amount of time between the stimulus and the qualified response. In some embodiments, the ear-worn device can be configured to detect a stimulus sufficient to generate a reaction from the ear-worn device wearer, monitor for a qualified response to the stimulus, and measure the amount of time between the stimulus and the qualified response.

Ear-worn devices are uniquely suited for measuring reaction time. In many cases, ear-worn devices are worn by a subject regularly and so reaction/reflex time trends can be calculated frequently and over extended periods of time to more accurate detect trends. Ear-worn devices can provide stimuli to the user in various ways including, but not limited to, auditory stimulation. Ear-worn devices herein can also include sensor packages to detect responses accurately and rapidly by virtue of being worn.

While the measurement of reaction/reflex time is usefully applied as an indicator of broader cognitive capacity, this metric is particularly useful when assessing a subject's falls risk. Slow reaction/reflex speed (e.g., large reaction time) is known to impede an individual from making timely postural adjustments to maintain stability, thereby leading to the subject to having a greater risk for falling. Therefore, embodiments herein can include determining fall risk, including using reaction/reflex time to allow for a more accurate determination of fall risk. Exemplary techniques of calculating fall risk are described in U.S. Publ. Pat. Appl. Nos. 2018/0233028, 2018/0228405, and 2018/0228405, the content of all of which is herein incorporated by reference. Embodiments herein can specifically include using reaction/reflex time as an input or parameter in determining/calculating a fall risk value or a fall risk threshold.

In addition, measures of reaction/reflex time can serve as in-situ measures of cognitive load and can be used to create individualized signal processing settings for the ear-worn device itself. Hearing impairment is known to increase the difficulty of receptive communication and can cause increased level of cognitive load. Various aspects of hearing devices can effectively decrease the amount of listening effort a wearer must expend to recognize and comprehend receptive communication (e.g., decrease cognitive load). Embodiments herein can include modifying hearing device configurations and/or creating individualized signal processing settings for the ear-worn device, the benefit of which can, in some cases, be determined through an observed decrease in reaction/reflex time. For example, in various embodiments, a hearing device configuration can be changed and/or a signal processing setting can be changed and then measurements of reaction/reflex time can be taken, with a decrease in reaction/reflex time being taken as indicative that the configuration change and/or signal processing setting change is beneficial for the device wearer.

Similarly, measures of reaction time herein can also be used to validate to new hearing device settings/features with users in the field. Therefore, embodiments herein can include validating hearing device settings/features for a subject or across a population of subjects in the field.

The term “response time” as used herein shall include reference to both reaction time and reflex time, unless the context dictates otherwise.

The term “ear-worn device” as used herein shall refer to devices that can aid a person with impaired hearing. The term “ear-worn device” shall also refer to devices that can produce optimized or processed sound for persons with normal hearing. Ear-worn devices herein can include hearing assistance devices. Ear-worn devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices. In some embodiments, the ear-worn device can be a hearing aid falling under 21 C.F.R. § 801.420. In another example, the ear-worn device can include one or more Personal Sound Amplification Products (PSAPs). In another example, the ear-worn device can include one or more cochlear implants, cochlear implant magnets, cochlear implant transducers, and cochlear implant processors. In another example, the ear-worn device can include one or more “hearable” devices that provide various types of functionality. In other examples, ear-worn devices can include other types of devices that are wearable in, on, or in the vicinity of the user's ears. In other examples, ear-worn devices can include other types of devices that are implanted or otherwise osseointegrated with the user's skull; wherein the device is able to facilitate stimulation of the wearer's ears via the bone conduction pathway.

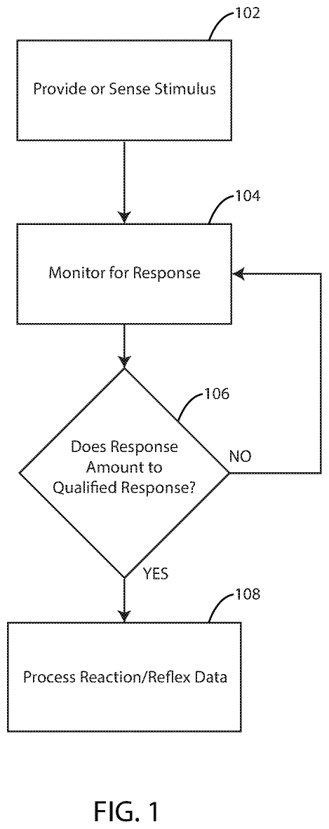

Referring now to FIG. 1, a flowchart of operations of various devices herein is shown in accordance with various embodiments herein. One operation includes providing (active mode) or sensing (passive mode) a stimulus 102. Thus, in some embodiments, the ear-worn device can operate in an active mode and itself delivers a stimulus and/or can cause another device to deliver a stimulus. Exemplary stimuli are discussed in greater detail below. Briefly, however, such stimuli can be audible, visual, tactile, and the like. The stimulus can be sufficient to generate a response from the ear-worn device wearer.

However, in some embodiments, the ear-worn device can operate in a passive mode wherein it does not itself deliver a stimulus or cause a stimulus to be delivered, but rather it detects an event which could serve as a stimulus. For example, the ear-worn device can sense an event likely to trigger a reflex or reaction, such as a loud sound (e.g., a sound exceeding a threshold value of loudness, such as a car door slamming, a car-horn, a doorbell, thunder, or the like) and/or a meaningful sound (such as the device wearer's name being spoken, a baby crying, an utterance matching an individual's voice selected from a group of predetermined individuals familiar to the device wearer, etc.). Threshold values of loudness of sensed stimuli are not particular limited but, in some embodiments, can be greater than or equal to 5 dB, 10 dB, 20 dB, 30 dB, 40 dB, 50 dB, 60 dB, 70 dB, 80 dB, 90 dB, 100 dB, 110 dB, 125 dB, 130 dB, or can be an amount falling within a range between any of the foregoing.

The ear-worn device can also sense directionality of possible stimuli. For example, the ear-worn device can be equipped with directional microphones that can determine the direction that sound is coming from. In some cases, microphones can be associated with separate devices and/or accessory devices and can be used to sense sound as well as determine directionality. For example, directionality of any stimuli can be determined with respect to horizontal degrees (wherein 0 degrees represents a direction directly in front of the device wearer and 180 degrees represents a direction directly behind a device wearer) and vertical angle (wherein 0 degrees represents a direction parallel with the direction of the face of the device wearer).

While not intending to be bound by theory, the passive mode can be particularly advantageous for subjects that do not want to be disturbed by testing procedures. The passive mode can also be very useful for longitudinal monitoring because of its limited burden on device wearers.

Another operation can include monitoring for a response 104. Monitoring for a response 104 can include gathering data/signals being generated by sensors, such as any of the sensors described in greater detail below. In some embodiments, monitoring for a response 104 can include activating one or more sensors that may not normally be active. In some embodiments, monitoring for a response 104 can include changing the rate of data acquisition of one or more sensors, such as a sampling rate and/or resolution of data. In some embodiments, monitoring for a response 104 can include saving data generated by one or more sensors in order to provide for later processing and evaluation of the same. In some embodiments, monitoring for a response 104 can include monitoring for a change in data/signals produced by one or more sensors. In some embodiments, monitoring for a response 104 can include transmitting data/signals produced by one or more sensors to a device and/or processor that is external to the ear-worn device.

The response may take various forms. The response can be a sound, such as the device wearer making a sound in response to the stimulus. The response can be detected movement. In some embodiments, the response can be a detected series of movements, such as a series of movements amounting to a balance recovery event/effort by the device wearer. The response can be a nerve impulse or nerve signaling (such as to detect the nerve signaling generating the post-auricular reflex, activation of periauricular muscles, tympanic reflex, acoustic reflex, vestibulo ocular reflex, startle response, and the like). The response can be indicative of a nerve signaling one of various other ocular movements, including nystagmus, ocular vestibular-evoked myogenic potential (OVEMP), sound-evoked vesitbulo-ocular reflex, and the like. Many other types of response are also contemplated herein.

Another operation can include evaluating 106 the detected response to determine if it amounts to an actual reaction or reflex (e.g., a “qualified response”). It will be appreciated that not all movements or other detectable actions amount to qualified responses to the stimulus. For example, a device wearer with Parkinson's may have a tremor causing a significant degree of spurious movement which is unrelated to a stimulus. Thus, in some embodiments, the ear-worn device can establish a baseline of normal movement (or typical range of normal movement) for an individual wearing the ear-worn device. Such a baseline can be generated during a calibration phase/mode. Such a baseline can be generated by evaluating data within the span of a moving window of time. The baseline data can include various statistical measures (e.g., mean, standard deviation, mode(s), variance, etc.) of normal movements or other signals in terms of magnitude, frequency, and the like.

In some embodiments, the evaluation can include evaluating 106 one or more of the magnitude of response (e.g., does the response cross a threshold magnitude), the directionality of the response (e.g., does the directionality of the response match any directionality of the stimulus), the timing of the response (e.g., does the response come at a time that is physiologically possible to qualify as a response), and the like. As an example of directionality of the response, a response can be a qualified response if a signal from a motion sensor indicative of the ear-worn device wearer turning their head toward a direction of a stimulus.

Another operation can include processing 108 data regarding the determined reaction or reflex. For example, processing 108 can include calculating one or more statistical values (e.g., mean, median, mode, deviation, etc.) relating to a response time based on the most recently detected response along with previously detected responses. In some embodiments, processing 108 can include calculating a change in response time over a most recent value, an average value, or a statistical value. In some embodiments, processing 108 can include comparing a response time against comparable device wearer response time(s), such as device wearers of similar age, sex, condition, etc. In some embodiments, the ear-worn device can be configured to evaluate the measured amount of time between the stimulus and the qualified response longitudinally and determine longitudinal trends. In some embodiments, the ear-worn device can be configured to evaluate the measured amount of time between the stimulus and the qualified response longitudinally and determine the impact of an event(s) occurring at a specific or generalized time, such as the effects of a brain injury or the improvement(s) realized from a treatment (e.g., use of a hearing device) or a therapy (e.g., aural rehabilitation, physical therapy, etc.). In some embodiments, the ear-worn device can issue an alert (perceptible by a human or another device) in response to a determined longitudinal trend or event crossing a threshold value.

In various embodiments, the ear-worn device or another device is configured to determine typical changes in the amount of time between the stimulus and the qualified response for the ear-worn device wearer. In various embodiments, the ear-worn device is configured to compare the amount of time between the stimulus and the qualified response for the ear-worn device wearer to one or more statistical measures relating to the amount of time between stimuli and qualified responses for a population ear-worn device wearers. In various embodiments, the ear-worn device is configured to determine typical amounts of time between the stimulus and the qualified response for a population of ear-worn device wearers based on the type of stimulus.

In some embodiments, processing 108 can include normalizing the data based on the type of stimulus. By way of example, the average reaction time for a visual stimulus is slower than the average reaction time for an auditory stimulus. Further, the average reaction time for an auditory stimulus can be slow then the average reaction time for a tactile stimulus.

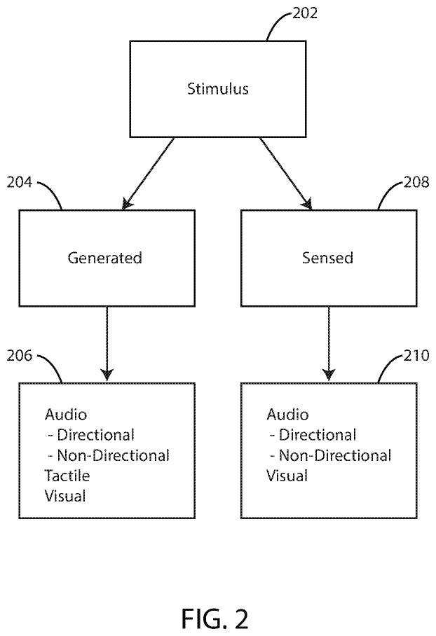

Referring now to FIG. 2, a diagram is shown illustrating types of stimuli used in accordance with various embodiments herein. As described above, the stimulus 202 can be a generated stimulus 204 or a sensed stimulus 208. Generated stimuli 204 can include, but are not limited to, exemplary generated 206 stimuli such as audio stimuli, tactile stimuli, visual stimuli, and the like. Exemplary sensed stimuli 210 can include, but are not limited to, audio stimuli, visual stimuli, and the like.

Stimuli herein can be directional or non-directional. By way of example, in some embodiments, an auditory stimulus can be provided without a particular direction. In some embodiments, an auditory stimulus can be provided that is perceived to originate from a particular direction.

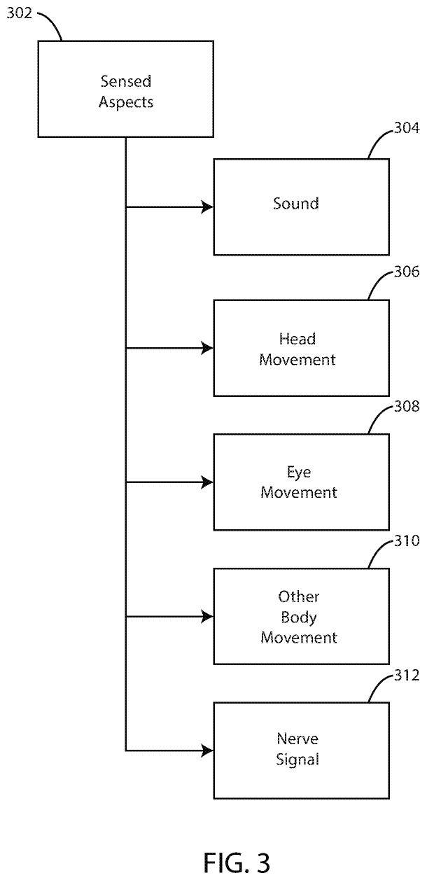

Referring now to FIG. 3, a diagram is shown illustrating some aspects that can be sensed as part of a possible response to a stimulus in accordance with various embodiments herein. As referenced above, the response may take various forms. The response can be a sound, such as the device wearer making a sound in response to the stimulus. The response can be detected movement. The response can be a nerve impulse or nerve signaling (such as to detect the nerve signaling generating the post-auricular reflex, activation of periauricular muscles, tympanic reflex, acoustic reflex, vestibulo ocular reflex, startle response, VOR, OVEMP, and the like). As such, sensed aspects 302 can include, but are not limited to, sound 304, movement, such as head movement 306, eye movement 308, and other body movement 310 (including movement of the ears through the post-auricular reflex, activation of periauricular muscles, movement of the arms, legs, hands, torso, etc.). Sensed aspects 302 can also include nerve signaling 312 (e.g., signals conducted through nerves), and the like. In some embodiments, sensed aspects 302 can also include physiological signals such as cardiac activity, blood pressure changes, breathing and breathing changes, and the like.

It will be appreciated that not every detected movement, nerve signal, etc. amounts to a response to a stimulus. As such, the sensed aspects must be evaluated to determine whether it is reflective of a response. In some cases, as described above, the evaluation can include evaluating 106 one or more of the magnitude of response, the directionality of the response (e.g., does the directionality of the response match any directionality of the stimulus), the timing of the response (e.g., does the response come at a time that is physiologically possible to qualify as a response, or does the response come at a time that is possible for the subject being evaluated), and the like.

In some cases, evaluation of a sensed aspect to determine whether it is reflective of a response can include performing statistical operations. For example, a set of previously measured response times (representing a certain number of prior response times, and/or prior response times falling with a previous time period) can be evaluated to determine a mean, a standard deviation, etc. It will be appreciated that various different mathematical and statistical operations may be applied to characterize a measured response time. In some embodiments, data or data signals may be compared between two or more sensors or types of sensors.

The previously measured response times can be from the same device wearer, or from a population of device wearers, such as a population of device wearers with similar characteristics. Then, the standard deviation (or another applicable statistic) can be used as a threshold for determining whether the sensed aspect is reflective of a response. For example, in various embodiments, a sensed aspect that is greater than 1, 1.5, 2, 2.5, 3, 3.5, or 4 standard deviations from the mean (or a number of standard deviations falling within a range between any of the foregoing) can be rejected as not being reflective of a response.

Various other methods can also be used for determining whether a sensed aspect is reflective of a response. For example, an interquartile range method can be used. In some embodiments, a percentile ranking method can be used with sensed aspects exceeding or falling below preselected percentile cutoff values being deemed to not reflect a response.

In some cases, the evaluation can also include characterizing the type of reaction/reflex detected. Reflexes are involuntary responses to a stimulus. Generally, reflexes can be faster than other types of responses. In various embodiments, reflexes can take less than 200, 100, 50, 40, 30, 25, 20, 15, 10, 5, 2.5, or 1 milliseconds. In some cases, reflexes can be fast because a reflex can be the result of motor neuron activation as initiated by an integrating center or interneuron in the spinal cord without requiring processing in the frontal lobes or motor cortex of the brain. As such, a detected response below a threshold value can be classified as a detected reflex.

Reactions are different than reflexes. Reactions can be slower than reflexes. Reaction times for healthy individuals can be from about 200 to 300 milliseconds (or depending on the type of response and the state of the individual, up to seconds). Reactions typically require the frontal lobe of the brain to evaluate signals and send instructions to the motor cortex of the brain, before motor control signals pass through the spinal cord and to the site of muscles being activated. It will be appreciated that other cortical areas may also be involved in generating reactions in response to stimuli, such as the temporal lobe, occipital lobe, and parietal lobe.

In some cases, certain stimuli may require a greater degree of cognitive processing (load) to select an appropriate response. By way of example, providing an auditory stimulus that instructs the user to turn to the left, but is delivered to be perceived as originating from the right side can be more likely to result in an increased cognitive load reaction. In some embodiments, the stimulus can be a dual-task paradigm stimulus (e.g., where attention must be divided between two concurrently performed task). Increased cognitive load reactions can be the slowest amongst responses (e.g., amongst reflexes, general reactions, and increased cognitive load reactions).

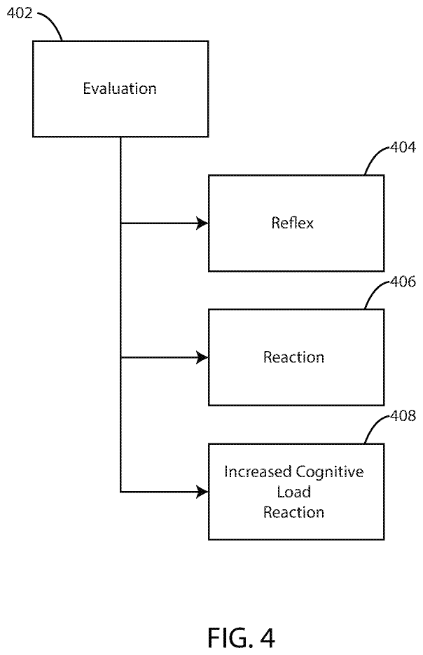

Referring now to FIG. 4, a diagram is shown illustrating aspects of response evaluation 402 in accordance with various embodiments herein. In various embodiments herein, response evaluation 402 can specifically lead to a conclusion of a reflex 404 being detected. In various embodiments herein, response evaluation 402 can specifically lead to a conclusion of a reaction 406 being detected. In various embodiments herein, response evaluation 402 can specifically lead to a conclusion of an increased cognitive load reaction 408 being detected.

In various embodiments, the type of response may also be influenced by the content/nature of the stimulus. For example, as referenced above, providing an auditory stimulus that instructs the user to turn to the left, but is delivered to be perceived as originating on the right side can be more likely to result in a cognitive processing reaction. As another example, a stimulus that is sufficient in magnitude to startle or exceed a comfort threshold is more likely to result in a reflex response.

Ear-worn devices herein, including hearing aids and hearables (e.g., wearable earphones), can include an enclosure, such as a housing or shell, within which internal components are disposed. Components of an ear-worn device herein can include a control circuit, digital signal processor (DSP), memory (such as non-volatile memory), power management circuitry, a data communications bus, one or more communication devices (e.g., a radio, a near-field magnetic induction device), one or more antennas, one or more microphones, a receiver/speaker, and various sensors as described in greater detail below. More advanced ear-worn devices can incorporate a long-range communication device, such as a BLUETOOTH® transceiver or other type of radio frequency (RF) transceiver.

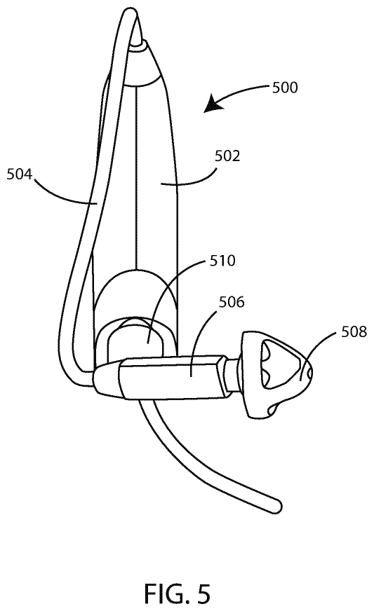

Referring now to FIG. 5, a schematic view of an ear-worn device 500 is shown in accordance with various embodiments herein. The ear-worn device 500 can include a hearing device housing 502. The hearing device housing 502 can define a battery compartment 510 into which a battery can be disposed to provide power to the device. The ear-worn device 500 can also include a receiver 506 adjacent to an earbud 508. The receiver 506 an include a component that converts electrical impulses into sound, such as an electroacoustic transducer, speaker, or loud speaker. Such components can be used to generate an audible stimulus in various embodiments herein. A cable 504 or connecting wire can include one or more electrical conductors and provide electrical communication between components inside of the hearing device housing 502 and components inside of the receiver 506.

The ear-worn device 500 shown in FIG. 5 is a receiver-in-canal type device and thus the receiver is designed to be placed within the ear canal. However, it will be appreciated that may different form factors for ear-worn devices are contemplated herein. As such, ear-worn devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices.

Ear-worn devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-worn devices of the present disclosure can employ other radios, such as a 900 MHz radio. Ear-worn devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files.

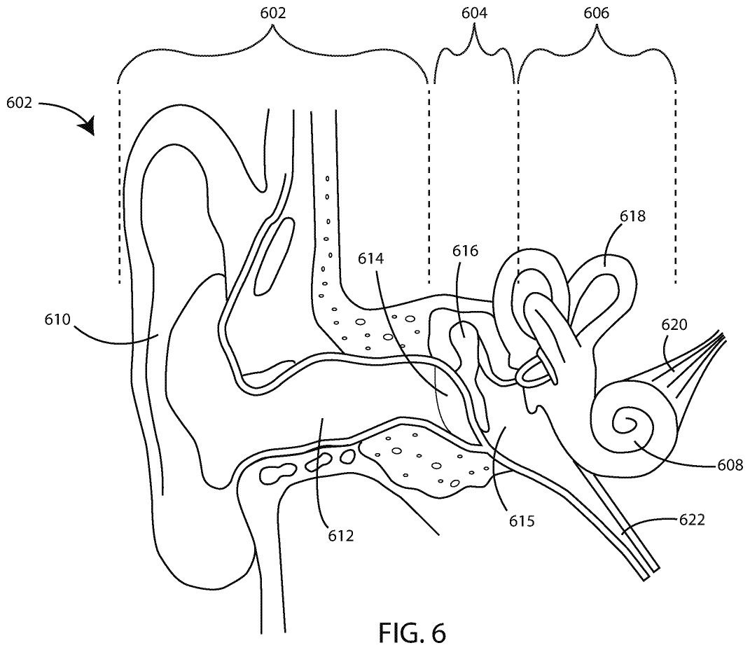

Referring now to FIG. 6, a partial cross-sectional view of ear anatomy is shown. The three parts of the ear anatomy are the outer ear 602, the middle ear 604 and the inner ear 606. The inner ear 606 includes the cochlea 608. (‘Cochlea’ means ‘snail’ in Latin; the cochlea gets its name from its distinctive coiled up shape.) The outer ear 602 includes the pinna 610, ear canal 612, and the tympanic membrane 614 (or eardrum). The middle ear 604 includes the tympanic cavity 615, auditory bones 616 (malleus, incus, stapes), and facial nerve. The inner ear 606 includes the cochlea 608, and the semicircular canals 618, and the auditory nerve 620. The pharyngotympanic tube 622 is in fluid communication with the eustachian tube and helps to control pressure within the middle ear generally making it equal with ambient air pressure.

Sound waves enter the ear canal 612 and make the tympanic membrane 614 vibrate. This action moves the tiny chain of auditory bones 616 (ossicles—malleus, incus, stapes) in the middle ear 604. The last bone in this chain contacts the membrane window of the cochlea 608 and makes the fluid in the cochlea 608 move. The fluid movement then triggers a response in the auditory nerve 620.

As mentioned above, the ear-worn device 500 shown in FIG. 5 can be a receiver-in-canal type device and thus the receiver is designed to be placed within the ear canal. Referring now to FIG. 6, a schematic view is shown of an ear-worn device disposed within the ear of a subject in accordance with various embodiments herein. In this view, the receiver 506 and the earbud 508 are both within the ear canal 612, but do not directly contact the tympanic membrane 614. The hearing device housing is mostly obscured in this view behind the pinna 610, but it can be seen that the cable 504 passes over the top of the pinna 610 and down to the entrance to the ear canal 612.

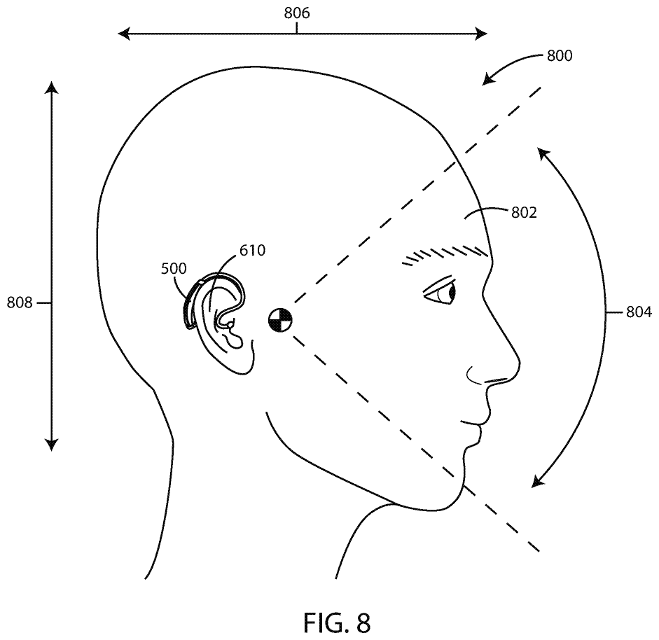

Ear-worn devices herein can include sensors (such as part of a sensor package 314) to detect movements of the subject wearing the ear-worn device. Referring now to FIG. 8, a schematic side view is shown of a subject 800 wearing an ear-worn device 500 in accordance with various embodiments herein. For example, movements detected can include forward/back movements 806, up/down movements 808, and rotational movements 804 in the vertical plane. Such sensors can detect movements of the subject and, in particular, movements of the subject's eyes, head, body, etc.

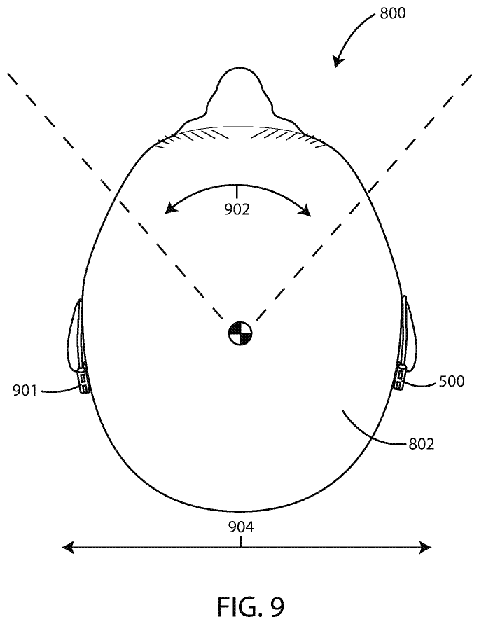

Referring now to FIG. 9, a schematic top view is shown of a subject 800 wearing ear-worn devices 500, 901 in accordance with various embodiments herein. Movements detected can also include side-to-side movements 904, and rotational movements 902 in the horizontal plane.

In accordance with various embodiments herein, the ear-worn device and/or the system can track movement of the subject's eyes using one or more of a camera, an EOG (electrooculogram) sensor, a VOG sensor, or another device. Movement of the subject's eyes can be used to identify a response (reflex or reaction). In some embodiments, a camera can serve as a motion sensor herein.

In various embodiments herein the ear-worn device itself provides a stimulus. However, in other embodiments an external device (e.g., external to the ear-worn devices) can provide the stimulus.

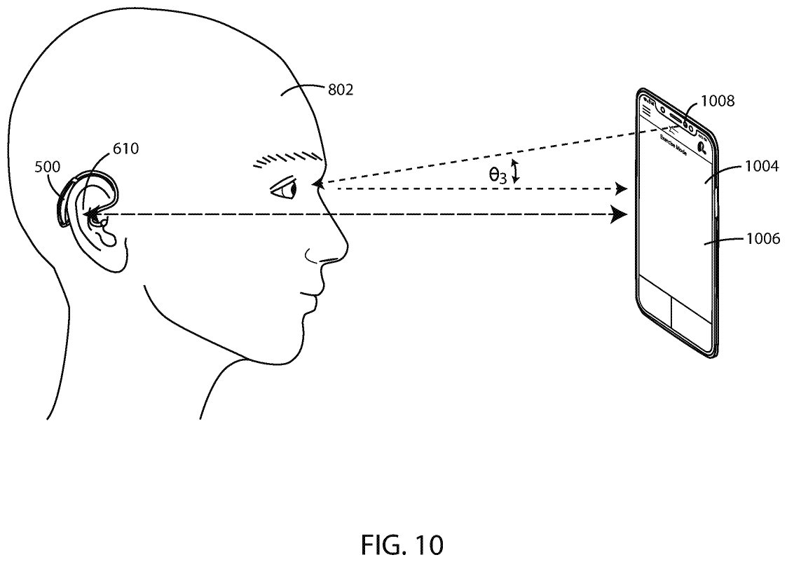

Referring now to FIG. 10, a schematic view is shown of device wearer 802 interfacing with an external device 1004 (which can be an external visual display device) in accordance with various embodiments herein. The external visual display device 1004 can include a display screen 1006 and a camera 1008. In some embodiments, the display screen 1006 can be a touch screen. The display screen 1006 can display various pieces of information to the subject 802 including, but not limited to, instructions for procedures to follow, visual feedback, a target or icon for the subject to focus their gaze on, information regarding the progress of the subject 802 through a particular set of procedures, or the like. In various embodiments, a visual stimulus can be provided through the display screen 1006. In various embodiments, an audible stimulus can be provided through speakers on the external device 1004. In various embodiments, a tactile stimulus can be provided by way of a vibration element inside the external device 1004.

The camera 1008 can be positioned to face toward the subject 802 (in some embodiments, the camera could also be facing the display, with the subject between the camera and the display screen—using the display itself as a spatial reference). The camera 1008 can be used to capture an image or images of the subject's 802 eyes. In some embodiments, the camera 1008 can be used to capture image(s) including the positioning of subject's 802 face, pupil, iris, and/or sclera. Such information can be used to calculate angle, speed and direction of eye movement, which can be evaluated to determine if it amounts to a response herein.

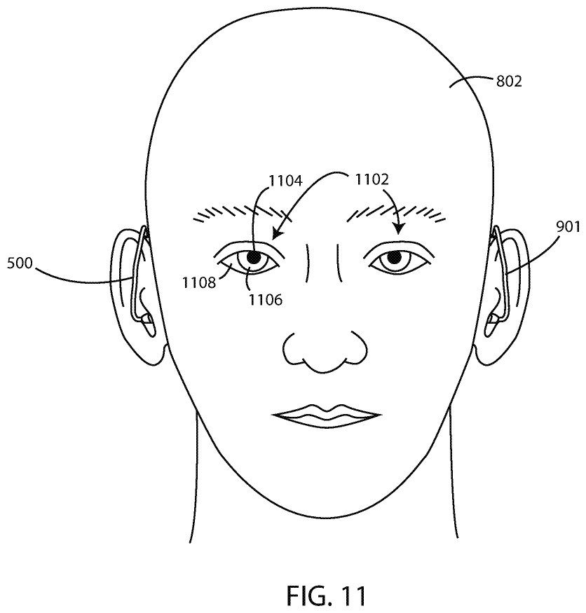

Referring now to FIG. 11, a schematic frontal view is shown of a subject 802 wearing ear-worn devices 500, 901 in accordance with various embodiments herein. The subject's 802 eyes 1102 include pupils 1104, iris 1106, and sclera 1108 (or white portion). Identifying the position of these and other eye components and facial components can be used to determine the direction of gaze and/or direction the face is pointing as described above.

In some embodiments, information from other sensors (such as an EOG sensor) can be used in combination with data from the camera to more accurately calculate the direction of the subject's face, gaze, eye movement or another aspect described herein. Aspects of EOG sensors are described in U.S. Pat. No. 9,167,356, the content of which is herein incorporated by reference in its entirety.

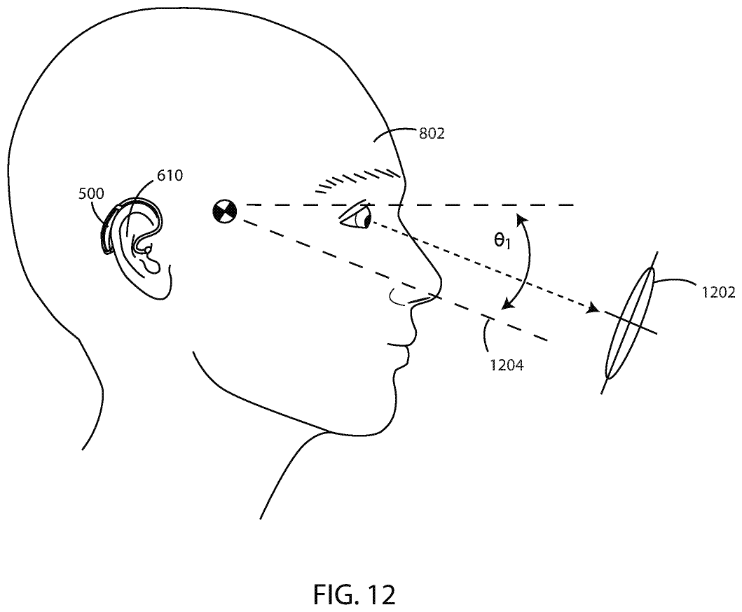

Referring now to FIG. 12, a schematic side view of a subject 802 wearing an ear-worn device 500 is shown in accordance with various embodiments herein. In this example, the subject 802 is prompted to direct their gaze at a target 1202. To comply with a stimulus, the subject (device wearer) 802 must tip (or rotate) their head downward causing the front of their face to be directed downward along line 1204, resulting in movement of angle θ1. In some embodiments, angle θ1 can be up to 5, 10, 15, 20, 25, 30, 35, 40, 50, 60, 70, or 80 degrees, or can be an angle that falls within a range wherein any of the foregoing can serve as the upper or lower bound of the range. Also, while this example shows simply rotation in a vertical plane, it will be appreciated that the target can also have horizontal rotational dimensions.

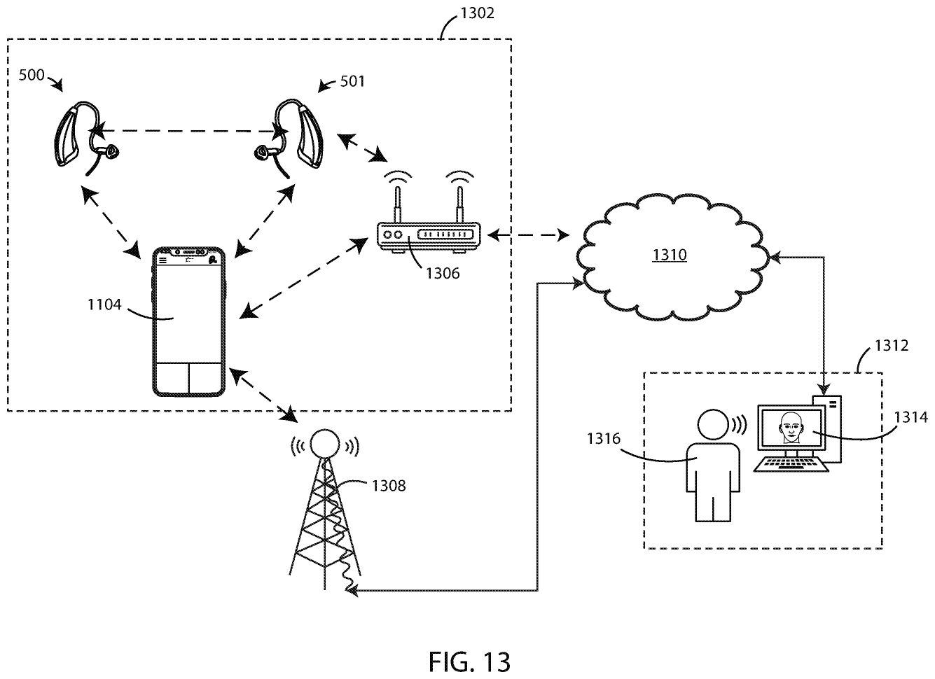

In various embodiments herein, measurements of response time (reaction or reflex time) can be reported back to an external data network and/or a third party. Thus, it will be appreciated that data and/or signals can be exchanged between many different components in accordance with embodiments herein. Referring now to FIG. 13, a schematic view is shown of data and/or signal flow as part of a system in accordance with various embodiments herein. In a first location 1302, a device wearer (not shown) can have a first ear-worn device 500 and a second ear-worn device 901. Each of the ear-worn devices 500, 901 can include sensor packages as described herein including, for example, an IMU. The ear-worn devices 500, 901 and sensors therein can be disposed on opposing lateral sides of the subject's head. In some embodiments, the ear-worn devices 500, 901 and sensors therein can be disposed in a fixed position relative to the subject's head. The ear-worn devices 500, 901 and sensors therein can be disposed within opposing ear canals of the subject. The ear-worn devices 500, 901 and sensors therein can be disposed on or in opposing ears of the subject. The ear-worn devices 500, 901 and sensors therein can be spaced apart from one another by a distance of at least 3, 4, 5, 6, 8, 10, 12, 14, or 16 centimeters and less than 40, 30, 28, 26, 24, 22, 20 or 18 centimeters, or by a distance falling within a range between any of the foregoing.

In various embodiments, data and/or signals can be exchanged directly between the first ear-worn device 500 and the second ear-worn device 901. An external visual display device 1004 with a video display screen, such as a smart phone, can also be disposed within the first location 1302. The external visual display device 1004 can exchange data and/or signals with one or both of the first ear-worn device 500 and the second ear-worn device 901 and/or with an accessory to the ear-worn devices (e.g., a remote microphone, a remote control, a phone streamer, etc.). The external visual display device 1004 can also exchange data across a data network to the cloud 1310, such as through a wireless signal connecting with a local gateway device, such as a network router 1306, mesh network, or through a wireless signal connecting with a cell tower 1308 or similar communications tower. In some embodiments, the external visual display device can also connect to a data network to provide communication to the cloud 1310 through a direct wired connection.

In some embodiments, a care provider 1316 (such as an audiologist, physical therapist, a physician or a different type of clinician, specialist, or care provider, or physical trainer) can receive information from devices at the first location 1302 remotely at a second location 1312 through a data communication network such as that represented by the cloud 1310. The care provider 1316 can use a computing device 1314 to see and interact with the information received. The received information can include, but is not limited to, information regarding the subject's response time (reaction time and/or reflex time). In some embodiments, received information can be provided to the care provider 1316 in real time. In some embodiments, received information can be stored and provided to the care provider 1316 at a time point after response times are measured.

In some embodiments, the care provider 1316 (such as an audiologist, physical therapist, a physician or a different type of clinician, specialist, or care provider, or physical trainer) can send information remotely from the second location 1312 through a data communication network such as that represented by the cloud 1310 to devices at the first location 1302. For example, the care provider 1316 can enter information into the computing device 1314, can use a camera connected to the computing device 1314 and/or can speak into the external computing device. The sent information can include, but is not limited to, feedback information, guidance information, and the like. In some embodiments, feedback information from the care provider 1316 can be provided to the subject in real time.

As such, embodiments herein can include operations of sending data to a remote system user at a remote site, receiving feedback from the remote system user, and presenting the feedback to the subject. The operation of presenting the auditory feedback to the subject can be performed with the ear-worn device(s). In various embodiments, the operation of presenting the auditory feedback to the subject can be performed with an ear-worn device(s).

Ear-worn devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-worn devices of the present disclosure can employ other radios, such as a 900 MHz radio or radios operating at other frequencies or frequency bands. Ear-worn devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

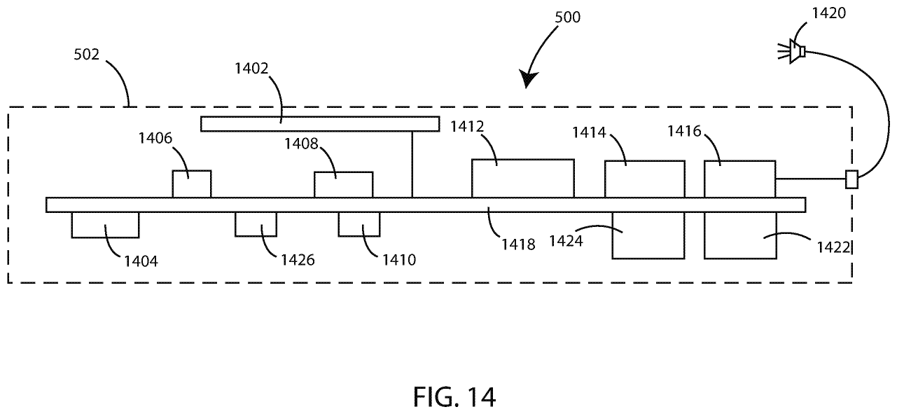

Referring now to FIG. 14, a schematic block diagram is shown with various components of an ear-worn device in accordance with various embodiments. The block diagram of FIG. 14 represents a generic ear-worn device for purposes of illustration. The ear-worn device 500 shown in FIG. 14 includes several components electrically connected to a flexible mother circuit 1418 (e.g., flexible mother board) which is disposed within housing 502. A power supply circuit 1404 can include a battery and can be electrically connected to the flexible mother circuit 1418 and provides power to the various components of the ear-worn device 500. One or more microphones 1406 are electrically connected to the flexible mother circuit 1418, which provides electrical communication between the microphones 1406 and a digital signal processor (DSP) 1412. Among other components, the DSP 1412 incorporates or is coupled to audio signal processing circuitry configured to implement various functions described herein. A sensor package 1414 can be coupled to the DSP 1412 via the flexible mother circuit 1418. The sensor package 1414 can include one or more different specific types of sensors such as those described in greater detail below. One or more user switches 1410 (e.g., on/off, volume, mic directional settings) are electrically coupled to the DSP 1412 via the flexible mother circuit 1418.

An audio output device 1416 is electrically connected to the DSP 1412 via the flexible mother circuit 1418. In some embodiments, the audio output device 1416 comprises a speaker (coupled to an amplifier). In other embodiments, the audio output device 1416 comprises an amplifier coupled to an external receiver 1420 adapted for positioning within an ear of a wearer. The external receiver 1420 can include an electroacoustic transducer, speaker, or loud speaker. The ear-worn device 500 may incorporate a communication device 1408 coupled to the flexible mother circuit 1418 and to an antenna 1402 directly or indirectly via the flexible mother circuit 1418. The communication device 1408 can be a BLUETOOTH® transceiver, such as a BLE (BLUETOOTH® low energy) transceiver or other transceiver(s) (e.g., an IEEE 802.11 compliant device). The communication device 1408 can be configured to communicate with one or more external devices, such as those discussed previously, in accordance with various embodiments. In various embodiments, the communication device 1408 can be configured to communicate with an external visual display device such as a smart phone, a video display screen, a tablet, a computer, or the like.

In various embodiments, the ear-worn device 500 can also include a control circuit 1422 and a memory storage device 1424. The control circuit 1422 can be in electrical communication with other components of the device. In some embodiments, a clock circuit 1426 can be in electrical communication with the control circuit. The control circuit 1422 can execute various operations, such as those described herein. The control circuit 1422 can include various components including, but not limited to, a microprocessor, a microcontroller, an FPGA (field-programmable gate array) processing device, an ASIC (application specific integrated circuit), or the like. The memory storage device 1424 can include both volatile and non-volatile memory. The memory storage device 1424 can include ROM, RAM, flash memory, EEPROM, SSD devices, NAND chips, and the like. The memory storage device 1424 can be used to store data from sensors as described herein and/or processed data generated using data from sensors as described herein.

It will be appreciated that various of the components described in FIG. 14 can be associated with separate devices and/or accessory devices to the ear-worn device. By way of example, microphones can be associated with separate devices and/or accessory devices. Similarly, audio output devices can be associated with separate devices and/or accessory devices to the ear-worn device.

### Stimuli

As described above, in various embodiments herein, one or more ear-worn devices can be used to provide a stimulus/stimuli to the device wearer and/or can issue an instruction to an external device (separate device) to provide a stimulus/stimuli to the device wearer. Also, in various embodiments, the stimulation can be sensed by the ear-worn device or a separate external device instead of being created by it (for example, a sensed ambient sound serving as a stimulus).

Many types of stimulation can be used herein. Stimulation can take the form of auditory stimulation, visual stimulation, tactile stimulation, nerve stimulation, electromagnetic field/radiation stimulation, and the like.

Depending on the type of stimulation provided, stimulation can be delivered to a selected site or sites of stimulation of the device wearer. Sites of stimulation herein can include, but are not limited to, on or about the ear, the ear canal, the inner ear, adjacent nerves related to the vestibular system, and the like. As one specific example, the ear canal can serve as a useful site of stimulation. In some embodiments, the site of stimulation can be at or adjacent to a distal end (innermost end) of the ear canal. In some embodiments, the site of stimulation can be at or adjacent to a proximal end (outermost end) of the ear canal. In some embodiments, the site of stimulation can be at a peripheral surface of the ear canal in between the distal and proximal ends. In some embodiments, the site of stimulation can be at or about the tympanic membrane. In some embodiments, the site of stimulation can be outside of the ear canal. In some embodiments, the site of stimulation can be on the ear itself. In some embodiments, the site of stimulation can be behind the ear. In some embodiments, the site of stimulation can be along the neck or brainstem.

Auditory stimulation can include the generation of sound delivered to the device wearer. In various embodiments, the volume, frequency, frequencies, frequency band or bands can be effective to achieve a stimulus that is likely to generate a response. In various embodiments, the auditory stimulation can be delivered at a frequency or frequency band within the bounds of normal human hearing (e.g., 20 to 20,000 hertz). In some embodiments, the auditory stimulation can be provided at multiple frequency bands. In some embodiments, the auditory stimulation can include sound with substantially equal volume across a broad frequency spectrum. In some embodiments, the auditory stimulation can include white noise at an intensity of at least about 60 dB SPL. In some embodiments, the auditory stimulation can include pink noise at an intensity of at least 60 dB SPL. In some embodiments, auditory stimulation can include audible words. In some embodiments, auditory stimulation includes no words, only sounds. In some embodiments, words as stimulation can be degraded. By way of example, in some embodiments, words can be time-compressed. In some embodiments, words can be vocoded speech. In some embodiments, an auditory stimulus can include competing noise with and/or frequency attenuation of the audible words.

Visual stimulation can include various types such as the flicker of a light in the environment of the device wearer and/or a particular graphic/pattern/symbol/color/text on a screen, such as on a smartphone, a TV, a tablet, a computer, a virtual reality, an augmented reality, a hologram, or the like. In various embodiments, the ear-worn device can send a command to a device with a screen to cause the visual stimulus to be shown and/or can send a command to a device to cause lights to flicker or turn off or on.

Tactile stimulation can include stimuli perceptible by receptors associated with the device's wearer's skin, epithelial tissue, or hair follicles such as free nerve endings, root hair plexus, Merkel's disks, Meissner's corpuscles, Ruffini corpuscles/endings, Pacinian corpuscles, lamalletad corpuscles, and the like. Tactile stimulation can include vibration, pressure, temperature changes, tension, and the like.

Nerve stimulation can include electrical stimulation of nerves. In some embodiments, a DC current can be applied. In some embodiments, an AC current can be applied. In some embodiments, the nerve stimulation can be applied for at least about 0.0001, 0.001, 0.01, 0.1, or 0.5 seconds or more, or an amount of time falling within a range between any of the foregoing. Exemplary electrical stimulation frequencies can include 5, 10, 20, 30, 40, 50, 60, 70, 80, 90, 100, 120, 140, 160, or 180 Hz, or frequencies falling within a range between any of the foregoing. Other frequencies are also contemplated herein.

Electromagnetic stimulation can include the generation of an electromagnetic field and/or electromagnetic radiation delivered to the device wearer. The frequency, frequencies, frequency band, frequency bands, waveform, amplitude, field strength, etc. can be effective to achieve a response. In some embodiments, the electromagnetic field can specifically be a magnetic field. In some embodiments, the electromagnetic field can be from about 1 to 30 kV/m in strength at a frequency of about 10, 20, 30, 40, 50, 60, 70, 80, 90, or 100 Hz (or falling within a range between any of the foregoing). In some embodiments, electromagnetic radiation can be applied at frequencies within the short radio wave band, long radio wave band, microwave band, and the like. In some embodiments, electromagnetic radiation can be applied at a power of 0.01, 0.1, 0.5, 1, 1.5, 2, 3, 4, 5, 7.5, 10, 20, 30, 40, or 50 Watts or more or an amount of power falling within a range between any of the foregoing.

In some embodiments, the stimulus can be a game event as part of a game. By way of example, a game can be played in which the device wearer receives points based on the speed of their responses and/or the number of responses they provide meeting minimum criteria. In some embodiments, the game event can be generated by the ear-worn device. In some embodiments, the game event can be generated by a separate device in electronic communication with the ear-worn device.

In some embodiments, the stimulus can include or be an action from the ear worn device or a separate external device requesting a response from the ear-worn device wearer. By way of example, the action can be a ringer sound, a message notification, or a query.

### Sensors

Ear-worn devices as well as medical devices herein can include one or more sensor packages (including one or more discrete or integrated sensors) to provide data. The sensor package can comprise one or a multiplicity of sensors. In some embodiments, the sensor packages can include one or more motion sensors amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GME, etc.) sensors may be used to detect motion or changes in position. In some embodiments, biometric sensors may be used to detect body motions or physical activity. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter, a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, the sensor package can be part of an ear-worn device. However, in some embodiments, the sensor packages can include one or more additional sensors that are external to an ear-worn device. For example, various of the sensors described above can be part of a wrist-worn or ankle-worn sensor package, or a sensor package supported by a chest strap.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-worn device. In addition to the external sensors discussed hereinabove, the sensor package can comprise a network of body sensors (such as those listed above) that sense movement of a multiplicity of body parts (e.g., arms, legs, torso). In some embodiments, the ear-worn device can be in electronic communication with the sensors or processor of another medical device, e.g., an insulin pump device or a heart pacemaker device.

### Methods

Many different methods are contemplated herein, including, but not limited to, methods of making ear worn devices, methods of using ear worn devices to detect reaction/reflex time, and the like. Aspects of system/device operation described elsewhere herein can be performed as operations of one or more methods in accordance with various embodiments herein.

In an embodiment, a method of measuring a response time of a hearing device wearer is included, the method including initiating the provision of a stimulus to the hearing device wearer with an ear-worn device. The ear-worn device can include a control circuit, a clock circuit in electrical communication with the control circuit, an electroacoustic transducer for generating sound in electrical communication with the control circuit, a power supply circuit in electrical communication with the control circuit, and monitoring for a qualified response to the stimulus using at least one of a motion sensor and a microphone.

In an embodiment, the method can include initiating the provision of a stimulus to the hearing device wearer can include delivering an auditory stimulus to the hearing device wearer.

In an embodiment, the method can include initiating the provision of a stimulus to the hearing device wearer can include delivering a tactile stimulus to the hearing device wearer. In an embodiment, the method can include initiating the provision of a stimulus to the hearing device wearer can include delivering a visual stimulus to the hearing device wearer. In an embodiment, the method can include initiating the provision of a stimulus to the hearing device wearer can include delivering an electrical stimulus to the hearing device wearer.

In an embodiment of the method, the qualified response can be motion detected with the motion sensor. In an embodiment of the method, the motion sensor is disposed within an ear worn device. In an embodiment of the method, the qualified response comprises a reaction motion. In an embodiment of the method, the qualified response comprises a post-auricular reflex or detected activation of periauricular muscles. In an embodiment of the method, the qualified response comprises a balance recovery event. In an embodiment of the method, the qualified response comprises sound detected with the microphone exceeding a threshold value.

As described above, measures of reaction/reflex time can serve as in-situ measures of cognitive load and can be used to create individualized signal processing settings for the ear-worn device itself. Embodiments herein can include modifying hearing device configurations and/or creating individualized signal processing settings for the ear-worn device, the benefit of which can, in some cases, be determined through an observed decrease in reaction/reflex time. For example, in various embodiments, a hearing device configuration can be changed and/or a signal processing setting can be changed and then measurements of reaction/reflex time can be taken, with a decrease in reaction/reflex time being taken as indicative that the configuration change and/or signal processing setting change is beneficial for the device wearer. Exemplary configuration elements and/or signal processing settings that can be changed can include, but are not limited to, one or more of amplification (gain) values at one or more frequencies (which can include bass/treble balance), compression thresholds, signal processing CODECs, speeds and knee points or ratios at one or more frequencies, delay settings at one or more frequencies, frequency shifting parameters/settings, noise reduction methods/settings/algorithms, speech enhancement methods, speech or tonal indictor volumes, and the like.

In some embodiments herein, a method can include changing at least one of a hearing device configuration or a signal processing setting and using the measured response time to determine if the change benefits the device wearer, wherein a decrease in the measured response time (as discrete measurement, average value, or other statistical measure) over a previously measured response time (as a discrete measurement, average value, or other statistical measure) is indicative of a benefit to the device wearer.

In some embodiments, the ear-worn device can be configured to evaluate the measured amount of time between the stimulus and the qualified response longitudinally and determine the impact of an event(s) occurring at a specific or generalized time, such as the effects of a brain injury or the improvement(s) realized from a treatment (e.g., use of a hearing device) or a therapy (e.g., aural rehabilitation, physical therapy, etc.). Thus, in some embodiments herein, a method can include evaluating a measured response time at a plurality of time points following an event or events (wherein the event can include, but is not limited to any of those described herein) and then determining the effect of the event or events on the device wearer through calculating a trend in measured response times and/or a comparison of measured response times (by way of averages or other statistical measures) before and after the event or events. In some embodiments, the event can be sensed by the ear-worn device. In some embodiments, the event can be sensed by a separate device. In some embodiments, the occurrence of the event can be input by a system user.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-worn device comprising
a control circuit;
a clock circuit in electrical communication with the control circuit;
a motion sensor in electrical communication with the control circuit;
an electroacoustic transducer for generating sound in electrical communication with the control circuit; and
a power supply circuit in electrical communication with the control circuit;
wherein the ear-worn device is configured to initiate generation of a stimulus sufficient to generate a response from the ear-worn device wearer; and
wherein the ear-worn device is configured to
monitor for a qualified response to the stimulus; and
measure an amount of time between the stimulus and the qualified response.

2. The ear-worn device of any of claims 1 and 3-26, further comprising a motion sensor in electrical communication with the control circuit.

3. The ear-worn device of any of claims 1-2 and 4-26, further comprising a microphone in electrical communication with the control circuit.

4. The ear-worn device of any of claims 1-3 and 5-26, the stimulus comprising an auditory stimulus.

5. The ear-worn device of any of claims 1-4 and 6-26, the stimulus comprising an auditory stimulus generated by the electroacoustic transducer.

6. The ear-worn device of any of claims 1-5 and 7-26, the stimulus comprising audible words.

7. The ear-worn device of any of claims 1-6 and 8-26, wherein the words are degraded.

8. The ear-worn device of any of claims 1-7 and 9-26, wherein the words are time-compressed.

9. The ear-worn device of any of claims 1-8 and 10-26, the stimulus further comprising competing noise, vocoded speech, and frequency attenuation.

10. The ear-worn device of any of claims 1-9 and 11-26, the stimulus comprising a tactile stimulus.

11. The ear-worn device of any of claims 1-10 and 12-26, the stimulus comprising a game event.

12. The ear-worn device of any of claims 1-11 and 13-26, the game event generated by a device in electronic communication with the ear-worn device.

13. The ear-worn device of any of claims 1-12 and 14-26, the stimulus comprising an action from an external device requesting a response from the ear-worn device wearer.

14. The ear-worn device of any of claims 1-13 and 15-26, the action comprising a ringer sound, a message notification, or a query.

15. The ear-worn device of any of claims 1-14 and 16-26, the stimulus comprising a dual-task paradigm stimulus.

16. The ear-worn device of any of claims 1-15 and 17-26, wherein the ear-worn device is configured to monitor for a qualified response using the motion sensor.

17. The ear-worn device of any of claims 1-16 and 18-26, wherein the qualified response comprises a reaction motion.

18. The ear-worn device of any of claims 1-17 and 19-26, wherein the qualified response comprises a post-auricular reflex or activation of periauricular muscles.

19. The ear-worn device of any of claims 1-18 and 20-26, wherein the qualified response comprises a balance recovery event.

20. The ear-worn device of any of claims 1-19 and 21-26, wherein the ear-worn device is configured to monitor for the qualified response using a microphone, an EOG sensor, or an EEG sensor.

21. The ear-worn device of any of claims 1-20 and 22-26, wherein the ear-worn device is configured to monitor for the qualified response using a sensor attached to a separate device.

22. The ear-worn device of any of claims 1-21 and 23-26, wherein the ear-worn device is configured to evaluate the measured amount of time between the stimulus and the qualified response longitudinally and determine longitudinal trends.

23. The ear-worn device of any of claims 1-22 and 24-26, wherein the ear-worn device issues an alert in response to a determined longitudinal trend crossing a threshold value.

24. The ear-worn device of any of claims 1-23 and 25-26, wherein the ear-worn device is configured to determine typical changes in the amount of time between the stimulus and the qualified response for the ear-worn device wearer.

25. The ear-worn device of any of claims 1-24 and 26, wherein the ear-worn device is configured to compare the amount of time between the stimulus and the qualified response for the ear-worn device wearer to an average amount of time between stimuli and qualified responses for a population ear-worn device wearers.

26. The ear-worn device of any of claims 1-25, wherein the ear-worn device is configured to determine typical amounts of time between the stimulus and the qualified response for a population of ear-worn device wearers based on a type of stimulus.

27. A method of measuring a response time of a hearing device wearer comprising:
initiating the provision of a stimulus to the hearing device wearer with an ear-worn device, the ear-worn device comprising
a control circuit;
a clock circuit in electrical communication with the control circuit;
an electroacoustic transducer for generating sound in electrical communication with the control circuit; and
a power supply circuit in electrical communication with the control circuit; and
monitoring for a qualified response to the stimulus using at least one of a motion sensor and a microphone.

28. The method of any of claims 27 and 29-40, wherein initiating the provision of a stimulus to the hearing device wearer comprising delivering an auditory stimulus to the hearing device wearer.

29. The method of any of claims 27-28 and 30-40, wherein initiating the provision of a stimulus to the hearing device wearer comprising delivering a tactile stimulus to the hearing device wearer.

30. The method of any of claims 27-29 and 31-40, wherein initiating the provision of a stimulus to the hearing device wearer comprising delivering a visual stimulus to the hearing device wearer.

31. The method of any of claims 27-30 and 32-40, wherein initiating the provision of a stimulus to the hearing device wearer comprising delivering an electrical stimulus to the hearing device wearer.

32. The method of any of claims 27-31 and 33-40, wherein the qualified response comprises motion detected with the motion sensor.

33. The method of any of claims 27-32 and 34-40, wherein the motion sensor is disposed within an ear worn device.

34. The method of any of claims 27-33 and 35-40, wherein the qualified response comprises a reaction motion.

35. The method of any of claims 27-34 and 36-40, wherein the qualified response comprises a post-auricular reflex or activation of periauricular muscles.

36. The method of any of claims 27-35 and 37-40, wherein the qualified response comprises a balance recovery event.

37. The method of any of claims 27-36 and 38-40, wherein the qualified response comprises sound detected with the microphone exceeding a threshold value.

38. The method of any of claims 27-37 and 39-40, further comprising using the measured response time to calculate a fall risk value or a fall risk threshold.

39. The method of any of claims 27-38 and 40, further comprising
changing at least one of a hearing device configuration or a signal processing setting; and
using the measured response time to determine if the change benefits the device wearer, wherein a decrease in the measured response time over a previously measured response time is indicative of a benefit to the device wearer.

40. The method of any of claims 27-39, further comprising
measuring the response time at a plurality of time points following at least one event;
determining whether the event has caused an improvement, a decline, or no change to the device wearer, wherein a longitudinal decrease in the measured response time is indicative of an improvement.

41. An ear-worn device comprising
a control circuit;
a clock circuit in electrical communication with the control circuit;
an electroacoustic transducer for generating sound in electrical communication with the control circuit;
a power supply circuit in electrical communication with the control circuit;
wherein the ear-worn device is configured to
detect a stimulus sufficient to generate a response from the ear-worn device wearer;
monitor for a qualified response to the stimulus; and
measure an amount of time between the stimulus and the qualified response.

42. The ear-worn device of any of claims 41 and 43-56, the stimulus comprising a detected auditory, tactile, or visual stimulus.

43. The ear-worn device of any of claims 41-42 and 44-56, the qualified response comprising a signal from a microphone.

44. The ear-worn device of any of claims 41-43 and 45-56, the stimulus comprising the ear-worn device wearer's name as detected with the microphone.

45. The ear-worn device of any of claims 41-44 and 46-56, the stimulus comprising an utterance matching an individual's voice selected from a group of predetermined individuals familiar to the device wearer.

46. The ear-worn device of any of claims 41-45 and 47-56, the stimulus comprising an action from an external device requesting a response from the ear-worn device wearer.

47. The ear-worn device of any of claims 41-46 and 48-56, the action comprising a ringer sound, a message notification, or a query.

48. The ear-worn device of any of claims 41-47 and 49-56, the qualified response comprising a signal from a motion sensor.

49. The ear-worn device of any of claims 41-48 and 50-56, the qualified response comprising a signal from the motion sensor indicative of at least one of eye movement, head movement, or a body movement.

50. The ear-worn device of any of claims 41-49 and 51-56, the qualified response comprising a signal from the motion sensor indicative of the ear-worn device wearer turning their head toward the direction of the stimulus.

51. The ear-worn device of any of claims 41-50 and 52-56, wherein the ear-worn device is configured to monitor for the qualified response using a sensor attached to a separate device.

52. The ear-worn device of any of claims 41-51 and 53-56, wherein the ear-worn device is configured to evaluate the measured amount of time between the stimulus and the qualified response longitudinally and determine longitudinal trends.

53. The ear-worn device of any of claims 41-52 and 54-56, wherein the ear-worn device issues an alert in response to a determined longitudinal trend crossing a threshold value.

54. The ear-worn device of any of claims 41-53 and 55-56, wherein the ear-worn device is configured to determine typical changes in the amount of time between the stimulus and the qualified response for the ear-worn device wearer.

55. The ear-worn device of any of claims 41-54 and 56, wherein the ear-worn device is configured to compare the amount of time between the stimulus and the qualified response for the ear-worn device wearer to an average amount of time between stimuli and qualified responses for a population ear-worn device wearers.

56. The ear-worn device of any of claims 41-55, wherein the ear-worn device is configured to determine typical amounts of time between the stimulus and the qualified response for a population of ear-worn device wearers based on a type of stimulus.
